(19) United States
(12) Patent Application Publication
(10) Pub. No.: US 2011/0257685 A1
(43) Pub. Date: Oct. 20, 2011

(54) PRE-STRESSED SPINAL STABILIZATION SYSTEM

(76) Inventors: J. Scott HAY, Parkland, FL (US); Ryan SINGH, Loxahatchee, FL (US); David Bradley JONES, Redding, CA (US); Nathanael Robert FERRARI, Redding, CA (US)

(21) Appl. No.: 13/087,757

(22) Filed: Apr. 15, 2011

Related U.S. Application Data
(60) Provisional application No. 61/324,600, filed on Apr. 15, 2010.

(51) Int. Cl. A61B 17/70; B29C 47/02; B29C 45/14

(52) U.S. Cl. 606/263; 606/264; 264/229

(57) ABSTRACT

A spinal stabilization system, including a spinal implant having an elongate polymer body; a wire embedded in the body, the wire straining the polymer body; and a mounting element coupled to the elongate polymer body to facilitate engagement of the body to a spinal segment; and an orthopedic anchor having a threaded shaft; a head coupled to the threaded shaft, the head defining a cavity therein; a prosthesis coupling element at least partially disposed in the cavity and movable with respect to the head; and at least one asymmetrical ring circumscribing a portion of the prosthesis coupling element.

### CROSS-REFERENCE TO RELATED APPLICATION

This application is related to and claims priority to U.S. Provisional Patent Application Ser. No. 61/234,600, filed Apr. 15, 2010, entitled “Spinal Fixation and Pedicle Screws,” the entirety of which is incorporated herein by reference.

### STATEMENT REGARDING FEDERALLY SPONSORED RESEARCH OR DEVELOPMENT

n/a

### FIELD OF THE INVENTION

The present invention relates to systems and methods of use thereof for orthopedic stabilization, and particularly, spinal stabilization.

### BACKGROUND OF THE INVENTION

Spinal fusion is considered the “gold standard” for surgically treating patients whose condition has become so severe and debilitated that conservative, non-surgical measures fail to provide relief. Using bone grafts along with implants such as metal plates, rods and screws, spinal fusion adjoins two adjacent vertebrae, thus stabilizing the segment and easing the patient's pain, numbness, weakness and/or lack of mobility. Recently, advances in spine surgery technology—including a greater focus on the principles of spinal load sharing—have led to significant advancements in the materials selected for spinal fusion implants or prostheses. In particular, the development of semi-rigid alternatives to replace the traditional metal rods used in the past has been undertaken in an effort to replicate the motion and loading characteristics of a healthy spinal segment. Such alternatives typically provide less rigidity than metal rods, with material characteristics more closely approximating that of natural bone. Approximating the natural biomechanics of a healthy spine segment or “motion preservation” aims to provide some degree of controlled motion that can, in part, prevent deterioration of adjacent discs experiencing increased forces and loading following a fusion procedure. A significant limitation, however, for non-metallic implants includes increased vulnerability to accelerated fatigue and resulting increased failure rates compared to metallic components.

In addition to motion preservation efforts, long-term success of a fusion procedure greatly benefits from bone ingrowth around the implanted prostheses. Achieving such bone growth is often difficult, as the implanted prostheses shield surrounding tissue from naturally occurring stresses and motion. Such stress shielding can result in tissue degradation, and reduce the overall health and condition of a treated spinal segment. Various approaches have been employed to stimulate bone growth, but they are not without their limitations. For example, stimulating bone growth may include using extra bone from a patient's pelvis (autograft), using bone and tissue from a donor (allograft), or using a manufactured bone substitute. However, such techniques maybe limited or undesirable due to the overall health of a patient (e.g., subjecting a patient to an additional procedure to procure bone tissue from another site on the patient); sterilization concerns of donor tissue; and/or availability of synthetic bone substitutes.

The promotion of bone growth has also been attempted from a hardware standpoint, but such micro-motion mechanisms typically require the implantation of additional components on an implanted pedicle screw or rod, which increases the overall complexity and cost of a surgical procedure. Accordingly, such hardware-based approaches have grown out of favor with hospitals and surgeons in recent times.

In view of the above limitations, it is desirable to provide a spinal stabilization system facilitating motion preservation of a spinal segment, providing a high degree of resistance to fatigue and cyclic loading associated with spinal segment forces, and promoting bone growth without adding to the complexity of an implantation procedure.

### SUMMARY OF THE INVENTION

The present invention advantageously provides a spinal stabilization system and methods of use and manufacturing thereof that facilitate motion preservation of a spinal segment, provide a high degree of resistance to fatigue and cyclic loading associated with spinal segment forces, and promote bone growth without adding to the complexity of an implantation procedure.

In particular, a spinal implant is provided, including an elongate polymer body; a wire embedded in the body, the wire straining the polymer body; and a mounting element coupled to the elongate polymer body to facilitate engagement of the body to a spinal segment. The wire may be metallic; may be constructed from at least one of Nitinol, cobalt, stainless steel, or titanium; may have a substantially circular cross-section; may have a substantially rectangular cross-section; and/or may compress at least a portion of the polymer body. The polymer body may be constructed from polyetheretherketone (PEEK) and may have an arcuate shape. The mounting element may define an aperture therethrough for engaging an orthopedic anchor.

An orthopedic anchor is provided, including a threaded shaft; a head coupled to the threaded shaft, the head defining a cavity therein; a prosthesis coupling element at least partially disposed in the cavity and movable with respect to the head; and at least one asymmetrical ring circumscribing a portion of the prosthesis coupling element. The anchor may further comprise a cap securing the prosthesis coupling element to the head; and/or a plurality of asymmetrical rings circumscribing a portion of the prosthesis coupling element, where at least one of the asymmetrical rings may define a first surface having an asymmetrical curvature and/or at least one of the asymmetrical rings may define a varying thickness. The prosthesis coupling element may define an elongated threaded portion extending from the head; and/or may be movable between approximately 0.001 inches and 0.010 inches from a centerline longitudinal axis defined by the head.

A method of manufacturing a spinal implant is provided, including applying a force to a wire; coupling a polymer to the wire through at least one of extrusion or injection molding processes; awaiting a time duration for the polymer to at least partially cure; and removing the force from the wire. The applied force may be between approximately 30% and 80% of an ultimate tensile strength of the wire.

Another method of manufacturing a spinal implant is provided, including inserting a wire into a substantially cured polymer body; applying a force to the wire; introducing a substantially uncured polymer onto the substantially cured polymer body; awaiting a time duration for the substantially uncured polymer to at least partially cure; and removing the force from the wire. Introducing the substantially uncured polymer onto the substantially cured polymer body may include overmolding the substantially uncured polymer onto the substantially cured polymer body.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an illustration of a perspective view of an example of a spinal stabilization system constructed in accordance with the principles of the present invention;

FIG. 2 is an illustration of a side view of the spinal stabilization system of FIG. 1;

FIG. 3 is an illustration of a top view of the spinal stabilization system of FIG. 1;

FIG. 4 is an illustration of a cross-sectional view of the spinal stabilization system of FIG. 1;

FIG. 5 is another illustration of a cross-sectional view of the spinal stabilization system of FIG. 1;

FIG. 6 is an illustration of an example of a ring of an example of a spinal stabilization system constructed in accordance with the principles of the present invention;

FIG. 7 is a side view of the ring in FIG. 6;

FIG. 8 is an illustration of an exemplary method of manufacturing a spinal prosthesis in accordance with the principles of the present invention; and

FIG. 9 is an illustration of another exemplary method of manufacturing a spinal prosthesis in accordance with the principles of the present invention.

### DETAILED DESCRIPTION OF THE INVENTION

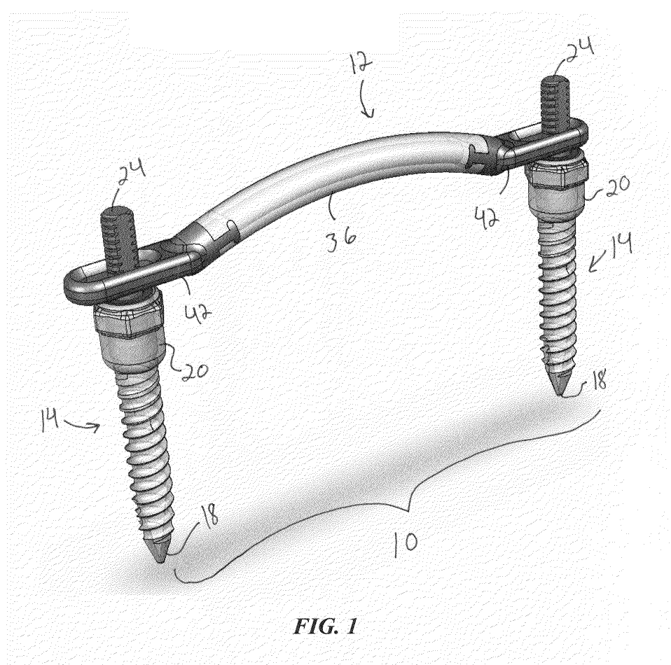

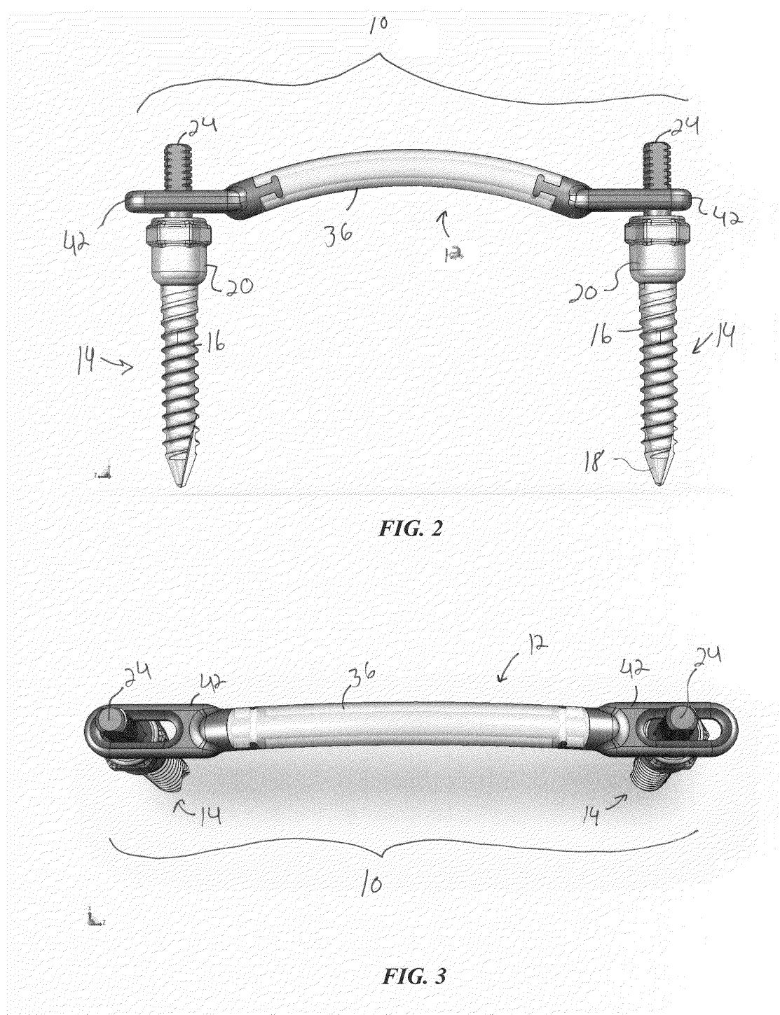

The present disclosure advantageously provides a spinal stabilization system and methods of use and manufacturing thereof that facilitate motion preservation of a spinal segment, provide a high degree of resistance to fatigue and cyclic loading associated with spinal segment forces, and promote bone growth without adding to the complexity of an implantation procedure. Referring now to the drawing figures in which like reference designations refer to like elements, an embodiment of a spinal stabilization system constructed in accordance with principles of the present invention is shown in FIGS. 1-5 and generally designated as “10.” The system 10 generally includes a spinal implant or prosthesis 12 engageable with one or more orthopedic anchors or screws 14. The spinal prosthesis may provide a desired degree of fusion, motion preservation, articulation, or the like depending on the particular application and patient's needs.

The one or more orthopedic anchors 14 may generally define or include a shaft 16 at least partially insertable or implantable into a targeted tissue region. The shaft 16 may include a threaded portion and a narrow or sharpened tip 18 to ease insertion. At an end of the shaft 16 opposite the tip 18, the anchor 14 may include a head 20 defining a cavity 22 therein. The cavity 22 may be dimensioned to receive a portion of an implant or prosthesis and/or intermediary structures facilitating engagement between the anchor 14 and an implanted prosthesis. For example, the anchor 14 may include a prosthesis coupling element 24 that is at least partly positionable within the cavity 22.

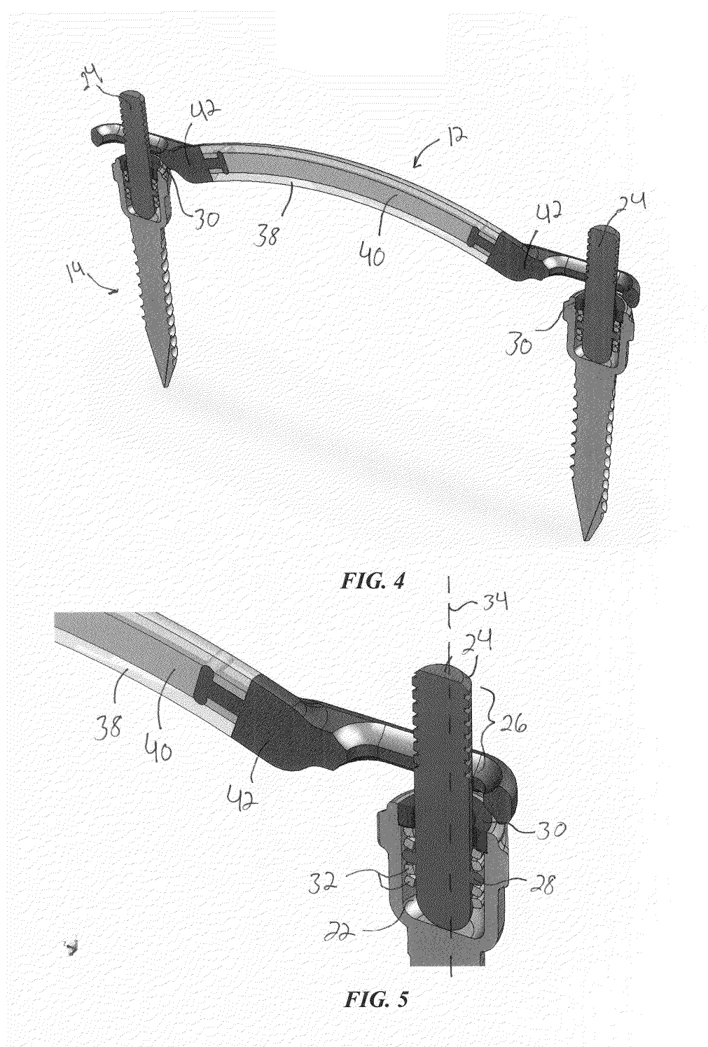

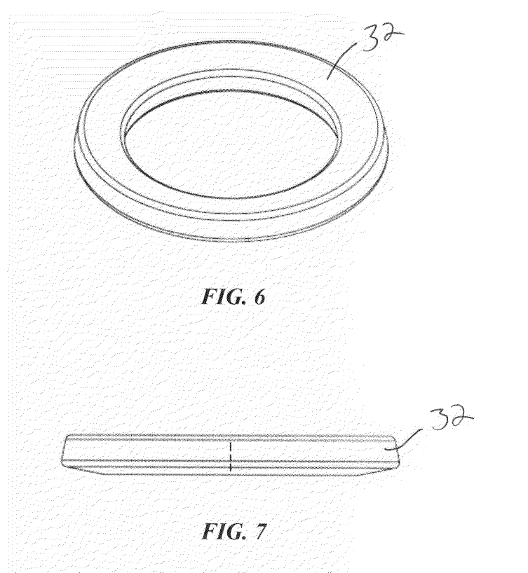

Referring now to FIGS. 4-5, the prosthesis coupling element 24 may be removable from the anchor 14, and may generally define an elongated, cylindrical shape partially disposed within the cavity 22, while also defining a length extending from the cavity 22 and away from the head 20. The portion of the prosthesis coupling element 24 extending outside of the head 20 may include a threaded portion 26 to allow a prosthesis to be coupled to the prosthesis coupling element 24, and securely fastened or clamped into position via the threads. The prosthesis coupling element 24 may further define or otherwise include a retention feature 28 that secures at least a portion of the prosthesis coupling element 24 within the head 20. For example, the retention feature 28 may include an annular ring or flange having a greater diameter than surrounding portions of the prosthesis coupling element 24, thus providing a ridge or shelf that can be secured within the head 20. The anchor 14 may include a cap or set screw 30 engageable with the head 20 to substantially secure the prosthesis coupling element 24 in place. The cap 30 may generally define a hole or aperture therethrough that is slidable or positionable around the prosthesis coupling element 24, while restricting passage of the retention feature 28.

The anchor 14 may provide a degree of motion between the anchor 14 and an attached prosthesis, and may further conduct or otherwise deliver stimulating motion into the surrounding tissue to promote tissue in growth. For example, the prosthesis coupling element 24 may be movable within or about the head 20 of the anchor 14, where such motion reverberates or is otherwise translated into micro stresses into the surrounding tissue to promote growth. Continuing to refer to FIGS. 4-5, the prosthesis coupling element 24 may be coupled to one or more annular rings or washers 32 that circumscribe a portion of the prosthesis coupling element 24 within the head 20, allowing for a limited range of motion or articulation between the prosthesis coupling element 24 and the head 20 and/or cap 30. For example, the one or more rings 32 may be irregular or asymmetrical such that a clearance between the prosthesis coupling element 24 and the head 20 (or cap 30) of the anchor 14 varies about different portions of the prosthesis coupling element 24, whether along its length and/or around its circumference or width. The one or more rings 32 may, for example, define an asymmetrical curvature on at least one surface to present a warped, bent, or otherwise deformed appearance or condition, as shown in FIGS. 6-7. The one or more rings 32 may, for example, define an asymmetrical cross-sectional width or thickness about one or more portions of the ring, and/or may be in the shape of a “conical donut” with an inner diameter or circumferential wall offset or skewed from an outer diameter or circumferential wall. The movable nature of the prosthesis coupling element 24 with respect to the head 20 may include an approximate range of motion between approximately 0.001 inches and 0.010 inches from a centerline longitudinal axis 34 defined by the head 20.

Referring again to FIGS. 1-3, the prosthesis 12 of the spinal stabilization system 10 may generally define an elongated body 36 that can span one or more segments of a spinal region and engage one or more orthopedic anchors, such as those described herein. As shown in FIGS. 4-5, the elongated body 36 may include a polymer layer or section 38 providing desired rigidity/flexibility characteristics approximating a healthy spinal joint and/or reducing stress shielding of affected tissues. For example, the polymer layer 38 may be constructed from polyether-etherketone (PEEK). PEEK is a radiolucent thermoplastic providing a high degree of biocompatibility, while also reducing the rigidity and associated stress-shielding of metallic implants. Though the elongate body 36 is shown spanning two anchors for an exemplary fusion approach, it is contemplated that one or more elongate bodies may be included coupled to one another with desired degrees of motion and/or articulation to provide dynamic stabilization or a desired range of motion for a treated spinal segment. The one or more elongate bodies may be coupled together to form a joint, telescoping movement, or the like across a single spinal joint or intervertebral disc, or alternatively, span a plurality of spinal joints.

The elongated body 36 may further include one or more wires 40 coupled to the polymer layer 38 to strain or otherwise exert a force on the polymer layer 38. For example, the one or more wire(s) 40 may exert a compressive force on at least a portion of the polymer section 38, thereby providing increased resistance to cyclical tensile stresses and bending associated with flexion/extension movement of the spine. The wire 40 may include a strand, filament, or tendon-like length of a material traversing substantially the entire length of the elongate body 36. The wire 40 may be constructed at least in part, from Nitinol, cobalt, stainless steel, titanium, carbon fiber, or the like. The wire 40 may have a substantially circular or substantially rectangular cross-section depending upon a particular application or desired biomechanical result. Further, the cross-sectional dimensions and/or percentage of the overall width of the elongate body 36 may vary by application and the desired amount of strain or pre-stress on the prosthesis. For example, the diameter of the elongate body 36 may range from approximately 4.0 mm and approximately 9.0 mm, while an example of a diameter of a wire 40 may range between approximately 0.05 mm to approximately 0.3 mm.

The prosthesis 12 may further include one or more mounting elements 42 coupled to the elongate body 36 to facilitate or aid in coupling the prosthesis 12 to one or more orthopedic anchors, such as one or more pedicle screws. For example, a mounting element 42 may be coupled to either end of the elongate body 36, and provide a plurality of mounting or coupling positions through an elongated opening or hoop. The mounting element(s) 42 may be embedded or fused to the polymer layer 38 and/or also coupled to the wire 40 of the elongate body 36. Though illustrated at both ends of the elongate body 36, it is contemplated that the mounting elements 42 may be positioned at other locations, such as a mid-length mounting point or lateral location adjacent to the elongate body 36. The mounting element(s) 42 may be constructed from a crush-resistant material, such as titanium, stainless steel or the like to reduce the likelihood of compromised structural integrity resulting from over-tightening or over-zealous securement of the prosthesis to an orthopedic anchor 14 or pedicle screw.

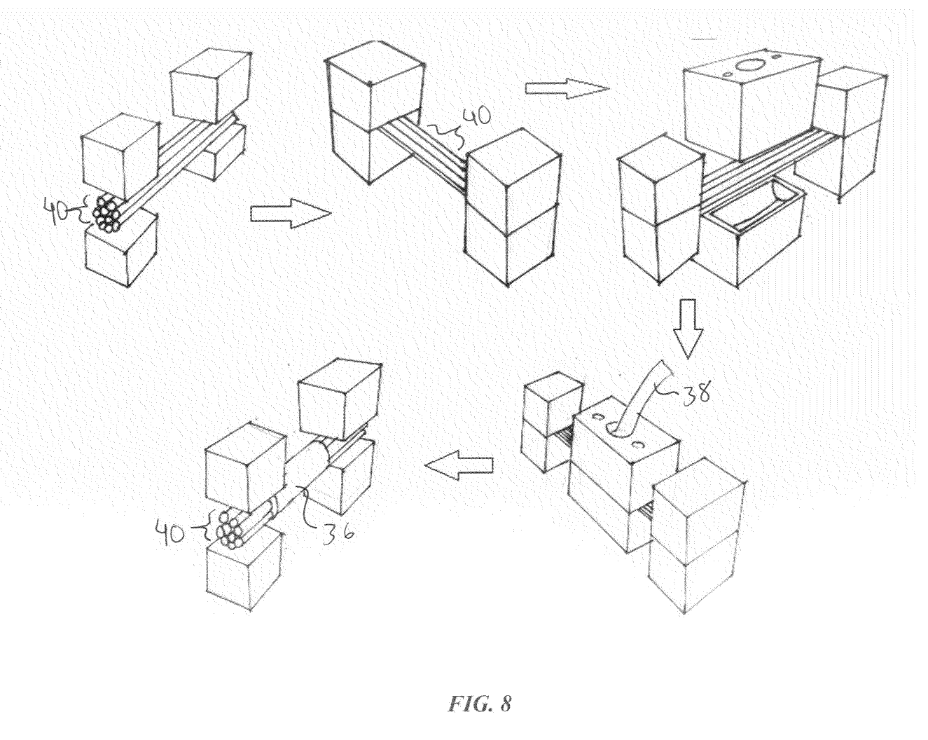

The pre-stressed configuration between the wire 40 and polymer layer or portion 38 of the elongate body 36 may be achieved by manufacturing techniques manipulating the wire 40 while one or more remaining portions of the elongate body 36 are formed or cured. For example, referring now to FIG. 8, one or more of the wires 40 may be attached or otherwise secured between two abutments, and a predetermined or preselected force may be applied to the wire(s) 40. The applied force may be calculated at least in part on the material properties of the wire 40, the desired resulting strain on the elongate body 36, the desired curvature (or lack thereof) for the prosthesis 12, or the like. For example, the force may be between approximately 30% and 80% of the wire's ultimate tensile strength. Alternatively, force may be applied to achieve a predetermined extension percentage of the overall length of the wire(s) 40. Once in their stretched or strained condition, the polymer layer or section 38 may be coupled to the wire(s) 40. The polymer layer 38 may, for example, be extruded or injection molded around the wire(s) 40 in a substantially uncured state, and the wire(s) 40 may remain subjected to tension for a time duration sufficient to achieve a substantially cured state of the polymer layer 38. Once the polymer layer 38 cures and/or reaches the desired strength, the tensioning forces on the wire(s) 40 may be released. As the wire(s) 40 react to at least partially regain their original state or length, tensile stresses are translated into a compressive stress on the polymer layer 38 of the elongate body 36. This method of manufacturing may be desirable for substantially linear elongate bodies to be used in regions of a spinal segment having minimal lordosis.

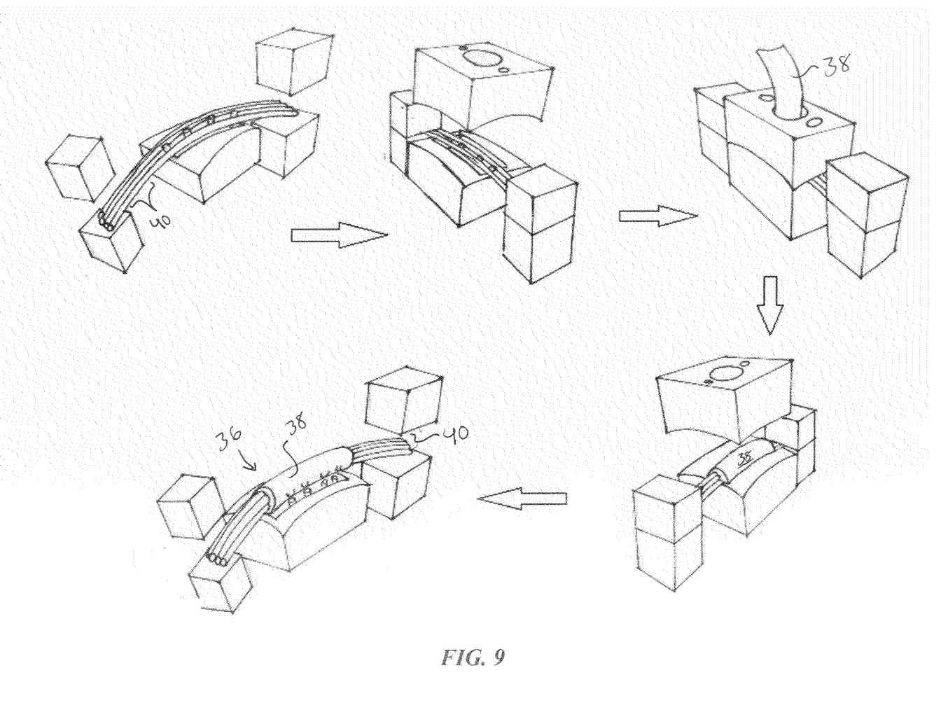

Alternatively, as shown in FIG. 9, the wire(s) 40 may be tensioned or otherwise subjected to force after a first polymer layer or body has cured satisfactorily. For example, a first polymer layer may be molded around or otherwise coupled to one or more of the wire(s) 40, where the coupling does not interfere with subjecting the wire(s) 40 to a selected strain or force. Cannulated polymer rods formed through extrusion or injection molding techniques may be employed, for example. The wire(s) 40 may be routed through the first polymer layer (such as a rod), and the one or more wire(s) 40 may then be subjected to a selected strain or elongation force against an end of the polymer layer and anchored off externally, placing the first polymer layer or section into compression. During a subsequent fabrication step, an over mold process may apply an additional layer of polymer material to secure the wire(s) 40 to the first polymer layer, and the force applied to the wire(s) 40 remains in place until the second polymer layer cures and/or reaches its desired strength. This method of manufacturing may be desirable for arcuate elongate bodies to be used in regions of a spinal segment having increased lordosis.

In an exemplary use of the spinal stabilization system 10, one or more of the orthopedic anchors 14 may be inserted into a spinal segment, such as in two adjacent vertebral discs or pedicles of a spinal joint. The prosthesis 12 may then be coupled to the one or more anchors 14. For example, the threaded portion 26 of the prosthesis coupling element 24 may be passed through the opening of the mounting element 42 of the prosthesis. Once the desired relative positions of the prosthesis coupling element 24 and mounting element 42 have been attained, a locking element such as a set screw or the like (not shown) may be fastened to the threaded segment 26 of the prosthesis coupling element 24 to lock the prosthesis 12 into place.

The spinal stabilization system provides increased tension resistance and thus increased prosthesis lifespan by implementing its pre-stressed configuration with the wire(s) and the one or more polymer layers. This decreased susceptibility to cyclic fatigue and failure avoids having to choose between a stabilization system that provides extended durations of use (e.g., such as with traditional exclusively metallic-based implants) and a system that provides increasingly desired biomechanical characteristics and motion preservation (e.g., such as with traditional exclusively polymer-based implants). Moreover, because of the articulation provided between the prosthesis coupling element and the head, growth-promoting stresses and movement are translated into the surrounding tissue to promote the overall health and longevity of the treated tissue area and the implanted system.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A spinal implant, comprising:
an elongate polymer body;
a wire embedded in the body, the wire straining the polymer body; and
a mounting element coupled to the elongate polymer body to facilitate engagement of the body to a spinal segment.

2. The implant of claim 1, wherein the wire is metallic.

3. The implant of claim 2, wherein the wire is constructed from at least one of Nitinol, cobalt, stainless steel, or titanium.

4. The implant of claim 1, wherein the polymer body is constructed from polyetheretherketone (PEEK).

5. The implant of claim 1, wherein the wire has a substantially circular cross-section.

6. The implant of claim 1, wherein the wire has a substantially rectangular cross-section.

7. The implant of claim 1, wherein the wire compresses at least a portion of the polymer body.

8. The implant of claim 1, wherein the elongate polymer body has an arcuate shape.

9. The implant of claim 1, wherein the mounting element defines an aperture therethrough for engaging an orthopedic anchor.

10. An orthopedic anchor, comprising:
a threaded shaft;
a head coupled to the threaded shaft, the head defining a cavity therein;
a prosthesis coupling element at least partially disposed in the cavity and movable with respect to the head; and
at least one asymmetrical ring circumscribing a portion of the prosthesis coupling element.

11. The anchor of claim 10, further comprising a cap securing the prosthesis coupling element to the head.

12. The anchor of claim 10, wherein the prosthesis coupling element defines an elongated threaded portion extending from the head.

13. The anchor of claim 10, further comprising a plurality of asymmetrical rings circumscribing a portion of the prosthesis coupling element.

14. The anchor of claim 10, wherein at least one of the asymmetrical rings defines a first surface having an asymmetrical curvature.

15. The anchor of claim 10, wherein at least one of the asymmetrical rings defines a varying thickness.

16. The anchor of claim 10, wherein the prosthesis coupling element is movable between approximately 0.001 inches and 0.010 inches from a centerline longitudinal axis defined by the head.

17. A method of manufacturing a spinal implant, comprising:
applying a force to a wire;
coupling a polymer to the wire through at least one of extrusion or injection molding processes;
awaiting a time duration for the polymer to at least partially cure; and
removing the force from the wire.

18. The method of claim 17, wherein the applied force is between approximately 30% and 80% of an ultimate tensile strength of the wire.

19. A method of manufacturing a spinal implant, comprising:
inserting a wire into a substantially cured polymer body;
applying a force to the wire;
introducing a substantially uncured polymer onto the substantially cured polymer body;
awaiting a time duration for the substantially uncured polymer to at least partially cure; and
removing the force from the wire.

20. The method of claim 20, wherein introducing the substantially uncured polymer onto the substantially cured polymer body includes overmolding the substantially uncured polymer onto the substantially cured polymer body.
